(19) United States
(12) Patent Application Publication
(10) Pub. No.: US 2006/0167480 A1
(43) Pub. Date: Jul. 27, 2006

(54) Anastomotic connectors

(75) Inventors: Amir Loshakove, Moshav-Bazra (IL); Ofer Nativ, Rishon-Lezion (IL); Ido Kilemnik, Herzelia (IL)

Correspondence Address: WOLF, BLOCK, SCHORR & SOLIS-COHEN LLP, 250 PARK AVENUE, NEW YORK, NY, 10177, US

(73) Assignee: By-Pass, Inc, Orangeburg, NY (US)

(21) Appl. No.: 10/529,110

(22) PCT Filed: Sep. 25, 2003
(86) PCT No.: PCT/IL03/00774

§ 371 Date: Mar. 24, 2005

(30) Foreign Application Priority Data
Sep. 25, 2002 (IL) IL02/00790

(51) Int. Cl. A61B 17/08

(52) U.S. Cl. 606/151

(57) ABSTRACT

Various anastomotic connectors (400) for attaching two blood vessels are described, including connectors, which comprise a plurality of clip like elements. In some embodiments, each clip like element comprises a flat medallion section (404) and a tearable hook section (402).

### RELATED APPLICATIONS

The present application claims priority from and is a continuation-in-part of PCT application PCT/IL02/00790, filed on Sep. 25, 2002, which designates the US, now published in English as WO 03/026475. It also claims priority as well as the benefit under 119 (e) of U.S. Ser. No. 60/492,998, filed on Aug. 7, 2003. This application is also a continuation-in-part of PCT/IL02/00215, filed on Mar. 18, 2002, PCT/IL01/01019, filed on Nov. 4, 2001, PCT/IL01/00903, filed on Sep. 25, 2001. The disclosure of all of these applications, which designate the US and were filed in English, are incorporated herein by reference.

### FIELD OF THE INVENTION

The present invention relates to anastomotic connectors.

### BACKGROUND OF THE INVENTION

Two blood vessels can be connected to form an anastomotic connection in many methods, including, for example, using surgical clips, using sutures, and using anastomotic connectors, for example as provided by Kaster in U.S. Pat. No. 5,234,447, the disclosure of which is incorporated herein by reference.

### SUMMARY OF THE INVENTION

A broad aspect of some embodiments of the invention relates to various types of anastomotic connectors and clips.

An aspect of some embodiments of the invention relates to a one piece clip adapted to form a part of an anastomotic connection, which clip is self locking. Optionally, the clip is pre-stressed so that when released, is closes and locks. Alternatively or additionally, the clip includes multiple locking positions.

An aspect of some embodiments of the invention relates to a one piece clip adapted to close and release from a delivery system when allowed to close, by the delivery system.

A broad aspect of some embodiments of the invention relates to locking two part connectors, in which an elongate hook section, having a hook at its end, is pulled through a medallion section and locked to the medallion section at its tip and the rest of the hook section removed, for example by tearing. In an exemplary embodiment of the invention, a plurality of two part connectors are used to complete an anastomosis, with each such connector acting as a clip. One potential advantage of not rigidly tying together the connectors is that the vessels of the anastomosis are not constrained by the such tying and are free to find a minimal stress-position and/or motion pattern.

An aspect of some embodiments of the invention relates to locking a two part anastomosis connector, in which tearing of an extension off of a hook section helps lock the hook section to a medallion section. In an exemplary embodiment of the invention, the tearing causes some parts of the hook section to distort and the hook section is configured so that the distortion causes a width of one section to be increased relative to a width of a passage in another section. In one example, the passage is narrowed. Alternatively or additionally, the width of the section is increased.

An aspect of some embodiments of the invention relates to a two part connector in which an elongate hook section includes a slot which engages a matching tab in a medallion section, which medallion section travels along the hook section. In an exemplary embodiment of the invention, the locking of the medallion section is within the slot. Alternatively or additionally, the locking of the medallion is on the outside of the hook section.

An aspect of some embodiments of the invention relates to a two part connector in which the hook section includes elements that move to effect the locking. Optionally, the medallion is passive with no parts thereof moving or substantially distorting for the locking. Alternatively, the medallion may have moving or distorting parts as well.

An aspect of some embodiments of the invention relates to a method of locking a hook section to a medallion where the hook section includes a first locking mechanism which engages a section of the medallion and where the medallion includes a second locking mechanism which engages a part of the hook section.

An aspect of some embodiments of the invention relates to a lock mechanism for a hook section, in which a widened section of the hook section includes an internal slot to allow the widened section to be compressed when a medallion section travels over it.

An aspect of some embodiments of the invention relates to providing a ring attachment on a medallion section. Optionally, the ring attachment may be used to increase a size of an aperture meant for a hook section, for example to assist in mounting the medallion on the hook section or to assist removal therefrom. Optionally, the ring attachment is adapted to be torn off, for example by pulling.

An aspect of some embodiments of the invention relates to a set of clip sections provided as a single connector in which staggered tearing times are provided. In an exemplary embodiment of the invention, the individual connectors are designed such that when tearing forces are applied, not all the connectors feel the forces at the same time. Thus, a smaller force needs to be applied to tear all the connectors. In one example, a single ring is used as a backing for all the medallions, However, each hook section has a different distance between the location where it is held by a pulling system and a location where the medallion is locked. The hook sections with the shortest distance, are torn first. Other variations may be provided, for example, different medallions may have different thicknesses or different hook sections may have different elongation properties or mechanical structures, allowing one hook section to elongate more before it tears, while a less-elongating hook section is torn before. Alternatively, the pulling system is skewed, pulling on some hook sections before others. The tearing may be designed, for example, to tear opposing legs together or serially, or to tear the legs in a different order, for example around the connection, possibly only one and possibly more than one leg at a time.

An aspect of some embodiments of the invention relates to an anastomotic connector in which a plurality of pullers form a part of a ring connector and pass inside the ring and, once released, pull blood vessel tissue towards the connector, to complete an anastomotic connection. Optionally, the pullers rotate alternatively or additionally to retracting. Optionally, the pullers pierce blood vessel tissue. Alternatively, the pullers do not pierce blood vessel tissue.

In an exemplary embodiment of the invention, the pullers are attached to a connector body via a curved section that describes an arc of greater than 270 degrees. Optionally, this curved section is long enough so that the pullers can have a considerable motion without exceeding elastic, super-elastic or shape memory properties of the material from which the connector is made. Optionally, the curved section flattens during the deployment.

There is thus provided in accordance with an exemplary embodiment of the invention, a self-locking clip adapted for vascular tissue connection, comprising:

a body;

a sharp extension on one side of the body and adapted to pierce blood vessel tissue; and

a base on another side of said body, said base adapted to interlock with said extension, at least to prevent retraction of said extension from said bas after inserted,

wherein said clip is pre-disposed to assume a closed configuration where said base locks to said extension. Optionally, said extension defines a plurality of locking positions. Alternatively or additionally, said base defines an aperture adapted to receive said extension. Optionally, said aperture is adapted to guide said extension to be locked.

In an exemplary embodiment of the invention, said clip is adapted to be used as part of a set of a plurality of clips to complete single anastomosis connection.

There is also provided in accordance with an exemplary embodiment of the invention, a clip delivery system, comprising:

a plurality of clips adapted for vascular tissue connection, each clip comprising:
a body;
a sharp extension on either end of said body,
wherein said clip is pre-disposed to form a “C” shape;

an inner tube defining a plurality of slots; and

an outer tube axially movable with respect to said inner tube,

wherein said inner and outer tube define a receptacle for said clip, said clip being released when said outer tube is retracted relative to said inner tube and wherein said inner tube defines a slot adjacent said receptacle, said slot adapted to receive a bent-back section of a backwards pointing one of said extensions. Optionally, said body defines an aperture and wherein said inner tube defines a matching protrusion to said aperture and wherein said body curves when released, such that said aperture is released from said protrusion.

There is also provided in accordance with an exemplary embodiment of the invention, a connector clip adapted for vascular tissue connection, comprising:

a hooked element comprising:
an elongate body;
a designated tear location structurally defined at a location along said body;
a hooked tip adapted to pierce a blood vessel; and

a base element adapted to lock to said hooked section at said locking location,

wherein said hooked element is adapted to not tear vascular tissue. Optionally, said hooked tip is adapted to not cut vascular tissue. Alternatively or additionally, said hooked tip has the shape of a needle. Alternatively or additionally, said hooked tip is manufactured by cutting and smoothing a planar material.

There is also provided in accordance with an exemplary embodiment of the invention, a connector clip adapted for vascular tissue connection, comprising:

a hooked element comprising:
an elongate body;
a designated tear location structurally defined at a location along said body;
a designated locking location structurally defined at a location along said body,
a hooked tip adapted to pierce a blood vessel; and

a base element adapted to lock to said hooked section at said locking location,

wherein said designated tearing location is configured to enhance a locking of said locking location. Optionally, said tearing location is configured so that tearing causes the bending of at least one part of said hooked element to narrow a passage of a portion of said base element within said hooked element. Alternatively or additionally, said tearing location is configured so that tearing causes the bending of at least one part of said hooked element to widen a portion of said hooked element which travels within said base element. Alternatively or additionally, said tip is adapted to pierce vascular tissue without causing tearing.

There is also provided in accordance with an exemplary embodiment of the invention, a connector clip adapted for vascular tissue connection, comprising:

a hooked element comprising:
an elongate body having a slot defined therein;
a designated tear location structurally defined at a location along said body;
a designated locking location structurally defined at a location along said body;
a hooked tip adapted to pierce a blood vessel; and

a base element adapted to lock to said hooked section at said locking location and including a section that fits in said slot,

wherein said designated locking location is located in said slot. Optionally, said hooked element comprises a second designated locking location on an outside of said hooked element. Alternatively or additionally, said tip is adapted to pierce vascular tissue without causing tearing. Alternatively or additionally, said base element is planar.

There is also provided in accordance with an exemplary embodiment of the invention, a connector clip adapted for vascular tissue connection, comprising:

a hooked element comprising:
an elongate body;
a designated tear location structurally defined at a location along said body;
a designated locking location structurally defined at a location along said body;
a hooked tip adapted to pierce a blood vessel; and

a base element adapted to lock to said hooked section at said locking location,

wherein said designated locking location is defined by at least one active portion on said hooked element which engages a portion of said base section. Optionally, said base element includes no portions that move relative to a center of gravity of said base element during a locking activity. Optionally, said base element is planar.

In an exemplary embodiment of the invention, said tip is adapted to pierce vascular tissue without causing tearing.

There is also provided in accordance with an exemplary embodiment of the invention, a connector clip adapted for vascular tissue connection, comprising:

a hooked element comprising:
an elongate body;
a designated tear location structurally defined at a location along said body;
a designated locking location structurally defined at a location along said body;
a hooked tip adapted to pierce a blood vessel; and

a base element defining a second locking location adapted to lock to said hooked element, wherein both said base element and hooked element each include at least one portion that moves during locking.

There is also provided in accordance with an exemplary embodiment of the invention, a connector clip adapted for vascular tissue connection, comprising:

a hooked element comprising:
an elongate body having an axis;
a designated locking location structurally defined at a location along said body;
a hooked tip adapted to pierce a blood vessel; and

a base element having an aperture adapted to ride on said body, said base element adapted to lock to said hooked element at said designated locking location,

wherein said locking location comprises at least one widening on said hooked element perpendicular to said axis and at least one aperture formed in said body adjacent said widening.

There is also provided in accordance with an exemplary embodiment of the invention, a connector clip adapted for vascular tissue connection, comprising:

a hooked element comprising:
an elongate body having an axis;
a hooked tip adapted to pierce a blood vessel; and

a base element having an aperture adapted to ride on said body and wherein at least one section of said base element contacting said aperture is adapted to be elastically moved to widen said aperture and comprising:
at least one holder adapted to widen said aperture when pulled against a resistance of said hooked element in said aperture. Optionally, said holder comprises an apertured holder. Optionally, said aperture contains a thread.

In an exemplary embodiment of the invention, said holder is adapted to be torn off said base element.

There is also provided in accordance with an exemplary embodiment of the invention, a method of mounting a base element of an anastomotic clip on a hooked element of an anastomotic clip, comprising:

placing said hooked element in an aperture of said base element; and

pulling on a holder section of said base element to widen said aperture.

There is also provided in accordance with an exemplary embodiment of the invention, a method of demounting a base element of an anastomotic clip on a hooked element of an anastomotic clip, comprising:

pulling on a holder section of said base element to widen an aperture of said base element on which said hooked element is mounted; and

removing said base element.

There is also provided in accordance with an exemplary embodiment of the invention, a connector clip set adapted for performing a vascular anastomotic connection, comprising:

a plurality of connector clips, each comprising:
a hooked element comprising:
   an elongate body having an axis, a first end and a second end;
   a pulling point adapted to have a pulling force applied to at said first end
   a hooked tip adapted to pierce a blood vessel at said second end
   a resting point for a base element between said ends; and
a base element adapted to ride on said body and stop at said resting point,

wherein a distance between said resting point and said pulling point is different for different ones of said clips. Optionally, said resting point is adapted to withstand a force of at least 1 Kg applied from said pulling point.

There is also provided in accordance with an exemplary embodiment of the invention, a pulling connector adapted for vascular tissue connection, comprising:

a ring; and

a plurality of hooked elements having an elongate body and curved into said ring,

wherein said elongate elements are pre-disposed to retract such that they pull vascular tissue towards said ring to complete an anastomotic connection. Optionally, said hooked elements rotate when released. Alternatively or additionally, said curves flatten when released.

In an exemplary embodiment of the invention, said hooked elements are adapted to pierce blood vessel tissue without tearing.

In an exemplary embodiment of the invention, said ring defines a plurality of recesses for said hooked elements.

In an exemplary embodiment of the invention, said ring is substantially rigid.

There is also provided in accordance with an exemplary embodiment of the invention, a method of deploying a tearing vascular anastomotic connector having multiple tearing points, comprising:

tearing a first leg to complete a first part of an anastomosis; and

tearing a second leg after said first tearing to complete a second part of said anastomosis. Optionally, the method comprises applying a continuous force to said connector during said first and second tearing and in between.

### BRIEF DESCRIPTION OF THE FIGURES

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1A is a perspective view of a closed self-locking clip, in accordance with an exemplary embodiment of the invention;

FIG. 1B is a side-cross-sectional view of a deployed clip forming a part of an anastomotic connection, in accordance with an exemplary embodiment of the invention;

FIGS. 2A-2C illustrate a process of using the clip of FIG. 1, in accordance with an exemplary embodiment of the invention;

FIG. 3 illustrates a delivery system for deploying a plurality of self-releasing clips, in accordance with an exemplary embodiment of the invention;

FIGS. 4A and 4B show plan views of a hook section of a hook-medallion connector, in accordance with an exemplary embodiment of the invention;

FIG. 4C shows a deployed hook-medallion connector, in accordance with an exemplary embodiment of the invention;

FIG. 4D shows a plurality of hook and medallion connectors, during deployment, in accordance with an exemplary embodiment of the invention;

FIGS. 5A and 5B show alternative forward parts of hook sections, in accordance with exemplary embodiments of the invention;

FIGS. 6A-6D show embodiments of hook sections with inner slots, in accordance with exemplary embodiments of the invention;

FIG. 6E shows a medallion section suitable for riding on a hook section of FIGS. 6A-6D, in accordance with an exemplary embodiment of the invention;

FIGS. 7A-7D show embodiments of medallion sections that ride on a slot of a hook section, in accordance with exemplary embodiments of the invention;

FIGS. 8A-8E show embodiments of medallions sections having locking to a hook section by one or more tabs of the medallion engaging the hook section from its front and/or back side, in accordance with exemplary embodiments of the invention;

FIGS. 9A-9C show medallions in which locking tabs lock into apertures defined in a side of a hook section, in accordance with an exemplary embodiment of the invention; and

FIGS. 10A-10E illustrate a puller connector and its use, in accordance with an exemplary embodiment of the invention.

### DETAILED DESCRIPTION OF EXEMPLARY EMBODIMENTS

Self Locking Clip

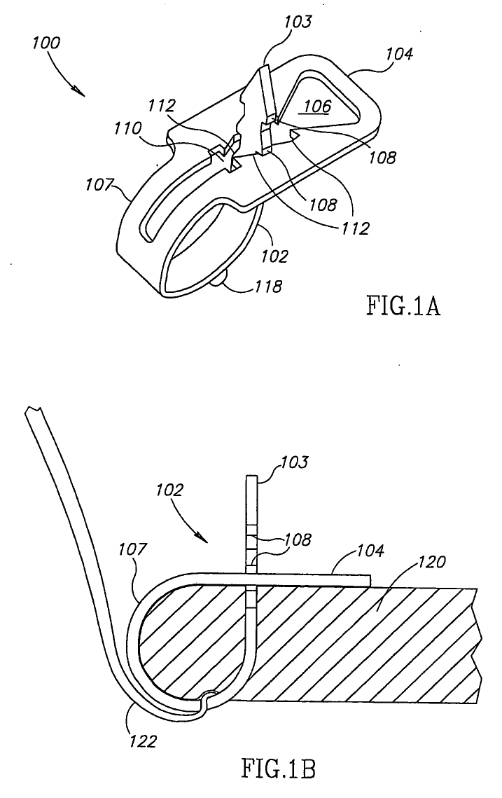

FIG. 1A is a perspective view of a closed self-locking clip 100, in accordance with an exemplary embodiment of the invention. FIG. 1B is a side-cross-sectional view of a deployed clip 100. Clip 100 comprises an extension 102 having a sharp point 103, that is adapted to pass through a target vessel 120, in a minimally-traumatic manner and pass through an aperture 106 in a base section 104 of clip 100. Optionally or additionally, aperture 106 defines one or more engagement areas 112 and/or extension 102 defines one or more engagement areas 108, such that clip 100 can lock, at least to prevent retraction of tip 103, in one or more locking positions.

In an exemplary embodiment of the invention, aperture 106 comprises a wide section marked with reference number 106 and a narrow section 110 where locking occurs. Optionally, the wider aperture section serves to define a part of base 104 as a spring section which is pre-disposed to oppose widening of slot 110. Optionally, slot 110 continues into a body section 107 of clip 100, that connect the base and extension sections of clip 100.

FIG. 1B is a cross-sectional view showing only one wall of a graft vessel 122 and part of a wall of aorta 120. FIG. 1B shows a particular eversion of graft vessel 122 on clip 100. However, in other embodiments, graft 122 need not be everted and may be, for example, transfixed on extension 102. The position of graft 122 relative to body 107 is optionally determined by an optional tissue stop 118. If no such tissue stop is provided and body 107 is narrow, then graft 122 may, for example, lie entirely outside of vessel 120.

A potential advantage of not having a tissue stop, which advantage may be practiced in other embodiments described herein if a tissue stop is not used, is that the force that a physician applies on the graft vessel can cause the vessel to selectively slide up. For example, if an oblique connection is desired, pulling the vessel into position may cause the vessel parts mounted on clips in the oblique angled section of the anastomosis to slide up, while vessel parts on the acute angled sections will not slide up. Such forces may be applied, for example, during the anastomosis or after it is completed.

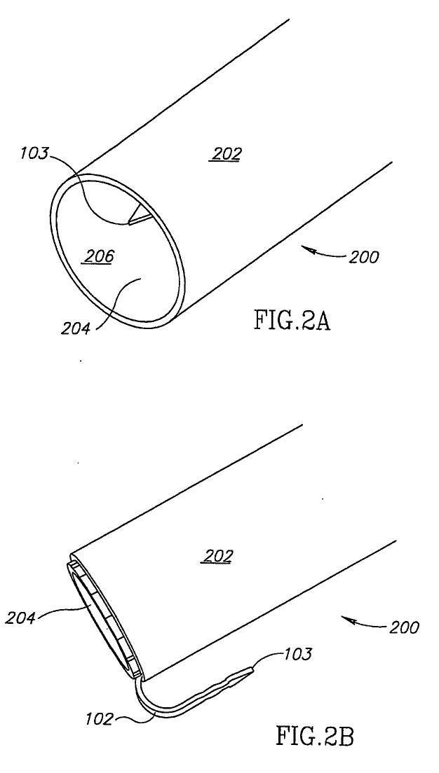

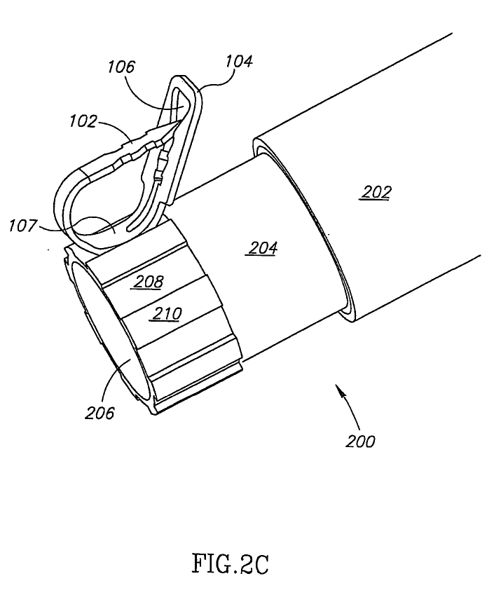

FIGS. 2A-2C illustrate a process of deploying clip 100, in accordance with an exemplary embodiment of the invention. While a plurality of clips are generally used, for clarity, only a single clip is shown.

In FIG. 2A, only a tip 103 of clip 100 protrudes from a delivery system 200. As will be clearer in the following figures, in an exemplary embodiment of the invention, clip 100 is held between an outer tube (visible) 202 and a mostly hidden inner tube 204. In an exemplary embodiment of the invention, a graft vessel is provided through delivery system 200, so that it exits via an aperture 206 at its end.

In an exemplary embodiment of the invention, the configuration of FIG. 2A is used for eversion, with a graft (not shown) being everted over tube 202 and, in the process, being transfixed by tips 103. It should be noted that even though clip 100 has a tendency (elastic, super elastic or shape memory, for example) to achieve the geometry of FIG. 2A, extension 108 is mostly straight, so that tip 103 does not bend away from the axis of tube 202.

In FIG. 2B, inner tube 204 is advanced, so that all of extension 102 is advanced forward and released from between tubes 202 and 204 and therefore, curves backwards. In an exemplary embodiment of the invention, this configuration is used for engaging a target vessel, for example an aorta. Delivery system 200 is placed into an aperture in the blood vessel, for example in the configuration of FIG. 2A, and extension 102 is released. Delivery system 200 is then pulled back, so that tip 103 engages the target vessel from inside. This step is considered complete when all of tips 103, of all the clips used are seen to protrude from the outside wall of the blood vessel. Alternatively, delivery system 200 may be inserted into the target vessel when already in the configuration of FIG. 2B, as there are no forward pointing sharp points or elements to interfere with such motion.

In FIG. 2C, tube 202 is further retracted, so that base section 104 of clip 100 is released and can lock to extension 102. A property of aperture 106 in accordance with some embodiments of the invention can be see, that aperture 106 is inclined so that if extension 102 lands in a wide section thereof and is pre-stressed to close towards body 107, the sides of aperture 106 will guide such closing.

Also visible in FIG. 2C is a mechanism for holding clips 100, in which a plurality of raised areas 210 define a plurality of recesses 208 between them. Each recess having the depth of the thickness of one clip and adapted to contain one clip. When tube 202 lies over tube 204, the clips are prevented from returning to their closed shape, except as they are released.

Self Releasing Clip

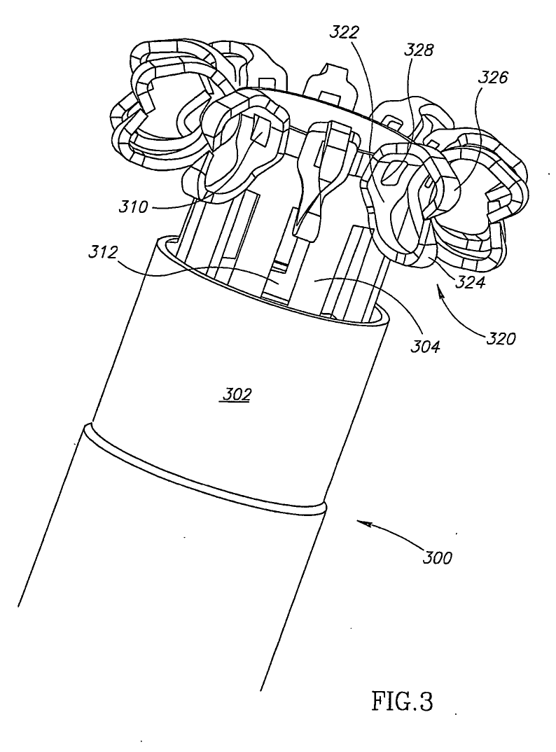

FIG. 3 illustrates a delivery system 300 for deploying a plurality of self-releasing clips 320, in accordance with an exemplary embodiment of the invention. Clips 320 are two sided clips having a body 322 with one extension on either end, 324 and 326. When released, as shown, the clips close to form a “C” shape. The deployment process can be as in FIGS. 2A-2C.

An exemplary delivery system 300 is shown, in which clips 320 are held between an inner tube 304 and an outer tube 302. In an exemplary embodiment of the invention, clips 320 are each maintained in an axial position by a projection 310 of inner tube 304, which fits into a recess or aperture 328 of clip 320. Optionally, when clip 320 is released, body 322 curves, curving aperture 328, thereby allowing projections 310 to slide by clips 320.

It should be noted that unlike clip 100, clip 320 has two curved extensions. In an exemplary embodiment of the invention, a plurality of slots 312 are provided in tube 304, so that extension 324 can be bend all the way back without distorting the clip in a manner which prevents deployment to the correct shape. In one example, when held by delivery system 300, the tip of extension 324 contacts the inner wall of tube 302, extension 324 is bent into slot 312 and the rest of clip 320 lies flat or is curved as does clip 100, in FIGS. 2A and 2B respectively. Optionally, tube 302 is coated on the inside with a softer material, such as a plastic, so that the point of extension 324, if made of metal, is not blunted.

Medallion and Hook Connector

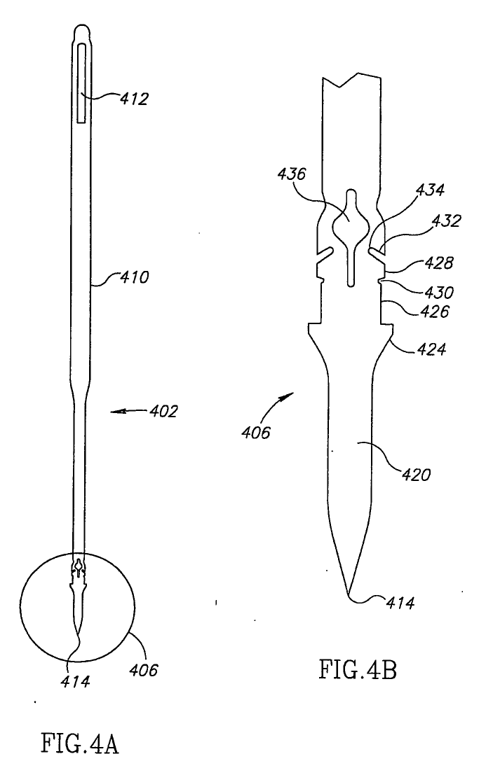

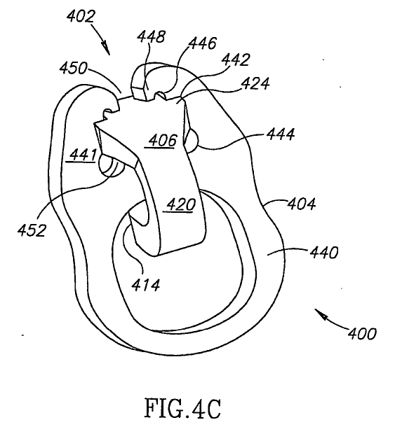

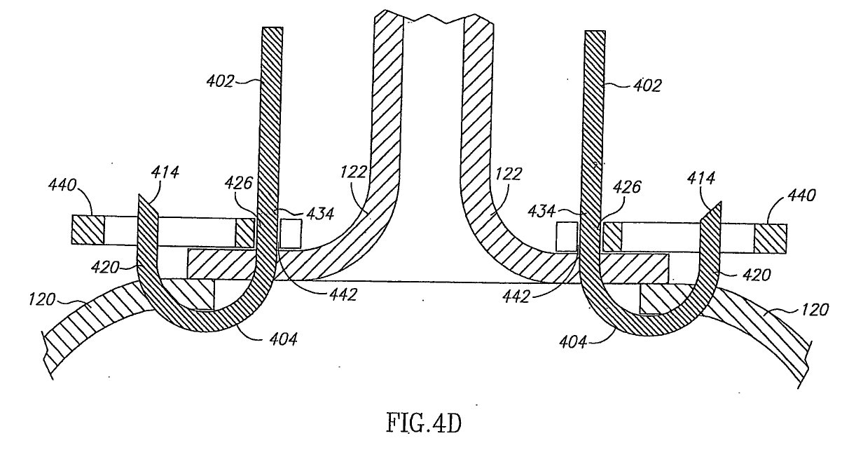

FIGS. 4A and 4B show a hook section 402 of a hook-medallion connector, in accordance with an exemplary embodiment of the invention. FIG. 4C shows a deployed hook-medallion connector 400, in accordance with an exemplary embodiment of the invention. FIG. 4D shows a set of connectors during deployment, prior to tearing thereof.

In a typically anastomosis, a plurality of hook-medallion connectors 400 are used, surrounding the anastomosis location. Each connector 400 comprises a hook section 402 and a medallion section 404. When deploying, a forward section 406 of hook section 402 engages and locks to medallion section 404 and the rest of hook section 402 is torn off and removed from the body.

Deployment may be similar, for example to that of FIGS. 2A-2C, except that instead of releasing a back section, hook sections 402 are retracted until they lock and tear. An exemplary delivery system is described in PCT publication WO 03/026475, the disclosure of which is incorporated herein by reference. After deployment, the connectors may be independent of each other, or be attached, for example by thin wires or sutures.

Referring in greater detail to FIG. 4A, hook section 402 comprises a body 410 having a slot 412 defined at one end thereof, and a tissue engaging tip 414 defined at another end thereof. Slot 412 is provided as an anchoring point for an engager (not shown) which retracts hook section 402 with sufficient force to tear it off of forward section 406. Tissue engaging tip 414 is shown as flat, however, when deployed, it is curved into a hook shape, as shown in FIG. 4C, for example. In an exemplary embodiment of the invention, the hook shape is configured so that it is about at the level of the plane of medallion 404, for example slightly below or slightly above. The exact position may depend on the amount of tissue trapped therein.

Referring in greater detail to FIG. 4B, forward section 106 comprises tip 414 and a tissue engaging section 420. Optionally, one or more tissue stops (e.g., increased width sections) are provided to control the slippage of tissue with respect to section 420. These stops are not show but may be positioned, for example between tip 414 and stop 424, described below. A locking area 426 is provided for locking to medallion section 404. One or more stop tabs 424 are provided to prevent forward motion of medallion 404. Optionally, tabs 424 are made strong enough to withstand the forces of tearing without substantial distortion, for example, forces of 1, 2.5, 5, 10, 20, 25 or more Kg. One or more back tabs 428 are provided to prevent medallion 404 from falling off of hook section 402. As shown, back tabs 428 are inclined in the forward direction and step-like in the backward direction. Optionally, the step-like structure makes reverse passage of medallion 404 difficult, while the inclined structure makes it easier. In an exemplary embodiment of the invention, a slot 436 is provided between tabs 428, so that the material of tabs 428 can be pressed into slot 436 while medallion 404 is passed over it One or more slots 432 are provided behind tabs 428, to define weakened areas 434, for tearing. Slot 436 optionally extends between two such weakened areas.

In an exemplary embodiment of the invention, slots 432 and weakened areas 434 are configured so that tearing forces will tend to cause tabs 428 to bend out rather than in. In the example shown, cut-out section 430 is provided to assist in such bending. The tearing action will generally first elongate and then tear. In the configuration shown, this will tend to cause tabs 428 to bend away from slot 436.

In an exemplary embodiment of the invention, tip 414 is manufactured so that it does not tear vascular tissue. While tip 414 may be made from a planar material, In an exemplary embodiment of the invention, it is smoothed, for example using electro-polish so that the only sharp section thereof is its needle like tip. Thus, it will tend to not tear vascular tissue when inserted thereto.

Referring in greater details to FIG. 4C, medallion section 404 comprises a ring section 440 through which tissue and tip 414 pass and a locking section 441 including an aperture 442 which rides on hook section 402. In the embodiment shown, aperture 442 is slightly undersized to receive hook section 402 and is thus elastically predisposed to engage hook section 402. In the embodiment shown, aperture 442 includes does not completely encircle hook section 402, an opening 450 is provided. Optionally, this allows a greater degree of movement for a pair of back tabs 448 which press against a back side of hook section 402. One or more removed sections 446 may be provided adjacent tabs 448, to define desired elastic properties of tabs 448. In general it should be noted that as a section is made thicker, a greater force is required to elastically deform it. Further, depending on the geometry of the section, a certain force may cause permanent distortion. Generally, sections that are longer have a greater distance they can be distorted elastically. In an exemplary embodiment of the invention, various removed sections are provided to control elastic forces and amount of movement without distortion, for example, a pair of removed sections 444 are provided adjacent a tab 452 on a front side of aperture 442. The removed sections sizes may be the same or may be different, depending, for example, on the desired properties. It should be noted that while a symmetric medallion is shown, an asymmetric design can be used as well. In an exemplary embodiment of the invention, medallion section 404 is substantially passive in that during locking and/or sliding, no parts of the medallion move relative to the center of gravity of the medallion. Optionally, any elastic ability of the medallion is to provide some leeway while mounting it on the hook sections or for applying a constant force against the hook section.

FIG. 4D shows a cross-sectional view of an exemplary anastomotic connection, after locking and prior to tearing.

The forces applied during tearing can be quite large, for example, 2.5 Kg per hook section, if all the hook sections are torn together, this might require a robust delivery system and/or cause movement by the user. In an exemplary embodiment of the invention, a connector set for an anastomosis is provided in which not all hooks are designed to bear tearing forces at a same time, thus a smaller force can be applied. Alternatively or additionally, the hook tearing may be staggered for other reasons, such as the ability to connect one side of the anastomosis first, for example for an oblique connection.

In some embodiments of the invention, the delivery system is designed to bear first on some hook sections and then on others. Alternatively, the hooks sections and/or medallion sections are designed to achieve a desired effect. In an exemplary embodiment of the invention, a set of connectors, for example arranged in a delivery systems is provided in which each hook has a desired relative tearing time.

In an exemplary embodiment of the invention, the distance between stop 424 and slot 412 is different for different hooks. The hooks with a shorter distance will feel a tearing force applied between those two points, first. Alternatively, slot 412 is made of different lengths for different hooks. Alternatively or additionally, different thickness medallions are provided. Alternatively or additionally, different elongation properties for different hook section are provided, for example, a bent ribbon section may be provided in one hook section to allow its greater elongation. Alternatively, chemical, mechanical and/or heat treatments are used to vary elongation.

Hook Section Variations

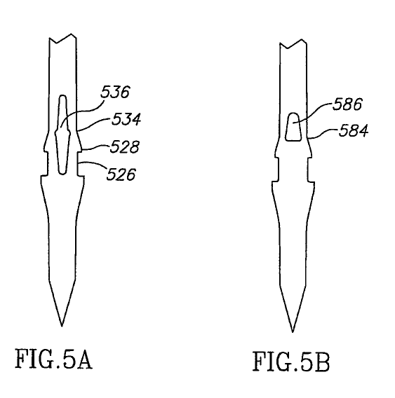

As can be appreciated, many variations on hook section 402 can be provided within the scope of the invention. for example, FIGS. 5A and 5B show alternative forward parts of hook sections, in accordance with exemplary embodiments of the invention. FIG. 5A shows an embodiment where a slot 536 underlies a locking area 526 as well as back locking tabs 528. Also, in this embodiment, a weakened area 534 is defined by cut outs on one or both sides, such as a widening of slot 536 and/or a narrowing of the body of the hook section from outside. Optionally, the part of slot 536 underlying locking area 526 is used for an inner locking of the medallion section, provided by a tab of the medallion section entering slot 536. Example medallions with a tab that locks to a slot underlying a locking area are described below. Alternatively or additionally, the elongated slot 536 increases the flexibility of back locking tabs 528. An elongated slot, however, may cause leakage of blood along the slot.

FIG. 5B shows a variation in which a slot 586 serves only to define a pair of weakened areas 584. This is an extreme example of a flexibility/sealing tradeoff, in that there will be no leakage when the slot is not in the blood vessel.

In another variation (not shown) a portion of the hook section is adapted to bend out of the plane of the hook and thus create an effective thickening of the hook. Such a thickening can also prevent reverse movement of the medallion on the hook.

Inner Slot Engagement

In FIGS. 4A-5B, the medallion section slides along the outside of the hook section. In some alternative embodiments, the medallion section at least engages a slot defined on the inside of the hook section

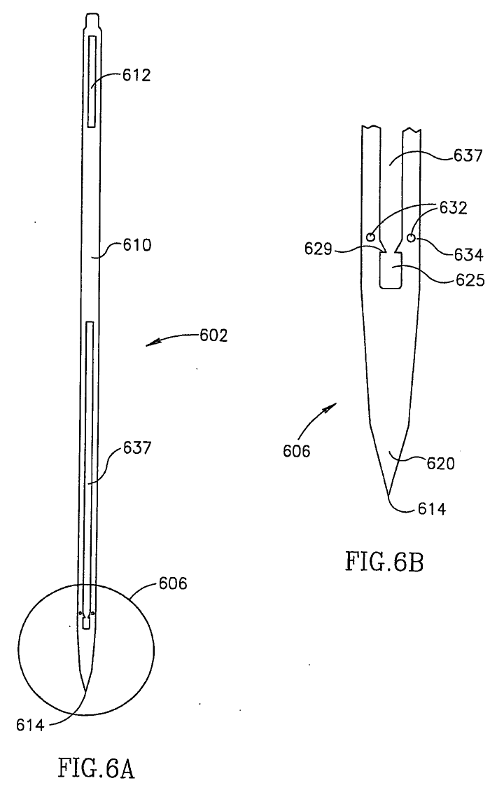

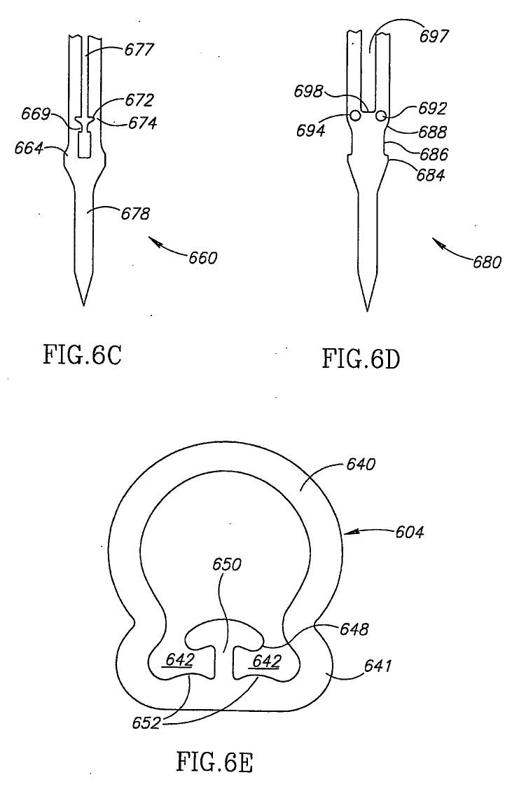

FIGS. 6A-6D show embodiments of hook sections with inner slots, in accordance with exemplary embodiments of the invention. FIG. 6E shows a medallion section suitable for riding on a hook section of FIGS. 6A-6D, in accordance with an exemplary embodiment of the invention.

Referring to FIG. 6A, a hook section 602 includes a body 610 with a forward section 606 shown in more detail in FIG. 6B. An optional back slot 612 is provided for engagement by a puller. Alternatively, a different structure, such as a “T” shaped end, may be used. A slot 637 for a medallion is provided along body 610 and into forward section 606.

Referring to FIG. 6B, forward section 606 has only inner locking mechanisms. Locking of a tab section of the medallion is provided in a locking area 625 which is a continuation of slot 637. One or more inner backwards tabs 629 are inclined so that forward motion along slot 637 is possible, while backwards motion once locking area 625 is reached, is difficult or impossible. Weakened areas 634 are optionally weakened by the provision of weakening apertures 632. It should be noted that in this and other embodiments, other methods of weakening may be used, for example, thinning, aperture forming, heat or chemical treatment and/or mechanical treatment.

While locking area 625 may have a length equal to the thickness of a medallion section, optionally, a greater length is provided, for example to allow the medallion some freedom of rotation and/or motion.

FIG. 6C shows a variant forward section 660, in which an external advancement stop 664 is provided to stop forward movement of the medallion and/or to stop advancement of tissue along section 660. A pair of inside backwards locking tabs 669 are shown for stopping reverse movement of a medallion out of a locking area 665. Optionally, the inclined nature of advancement stops 664 serves the purpose of providing an elastic stop whereby if the force applied to the medallion is sufficient, a small advancement is possible.

A pair of weakened areas 674 are defined by a widening 672 in slot 677. It should be noted that when tearing weakened areas 674, locking tabs 669 will tend to bend inwards.

In the particular embodiment shown, slot 677 is narrower than slot 665. In other embodiments, they may be the same width or slot 677 may be wider. Optionally, for example as will be shown below for some designs of medallions, a first tab section of the medallion rides in slot 677 and when slot 677 widens into slot 665, additional tab sections of the medallion enter slot 665. Optionally, for some medallion designs, no parts of the medallion are ever inside slot 677, except for locking area 665, when locking occurs.

Another difference which can be noted between section 606 and section 660, is that a body 620 of FIG. 6B is thinner than a body 678 of FIG. 6C. This variation may depend, for example, on the robustness of the blood vessels being attached and/or the size of the anastomosis.

FIG. 6D shows a forward section variation 680, in which a locking area 686 is defined between at least one forward outside tab 684 and at least one backward outside tab 688. A weakened area 694 is defined by a weakening aperture 692. Optionally, a slot 697 continues (not shown) adjacent locking area 686, thereby providing an inner locking area. Alternatively, in the embodiment shown, a section of the medallion stays in slot 697, against an end 698 thereof. Alternatively, slot 697 is used for the tearing mechanisms. As can be appreciated, the forces applied during tearing are considerable and might conceivably damage the connector. In an exemplary embodiment of the invention, tearing forces are not applied to the medallion or to locking area 686. Instead, a tab of the deliver system rides in slot 697. Tearing is achieved by pulling back on the hook connector from its far end (e.g., 612, FIG. 6A) while maintaining the tab in place against end 698.

FIG. 6E shows a medallion 640 including a tab section 650 which can ride along a slot in a hook section, such as slot 637. While a ring section 640 is shown, it should be noted that in some applications this ring section can be replaced by a sharp extension adapted to impale tissue, for example, an extension of tab 650 bent into the figure plane towards the tip of the hook section. More examples of such a design and other clips designs are provided in a PCT application filed by a same applicant “By-Pass inc.”, in the Israel receiving office, on same date as the instant application, having a title of “Sliding Surgical Clip” and having an attorney docket number of 088/03506, the disclosure of which is incorporated herein by reference. Furthermore, while a locking section 641 is shown, in some embodiments of the invention, this section is dispensed with and riding and locking are provided by tab 650. In the embodiment shown, however, a pair of optional forward locking tabs 648 are shown which urge against parts of the hook section that ride in an area 642 adjacent tabs 648. Alternatively or additionally, backwards tabs 652 are provided for a similar function. Locking area 641 optionally functions as an elastic spring to urge the tabs against the hook section, optionally into apertures or cut-outs defined in the hook section for that purpose.

Slot Riding Medallion Variations

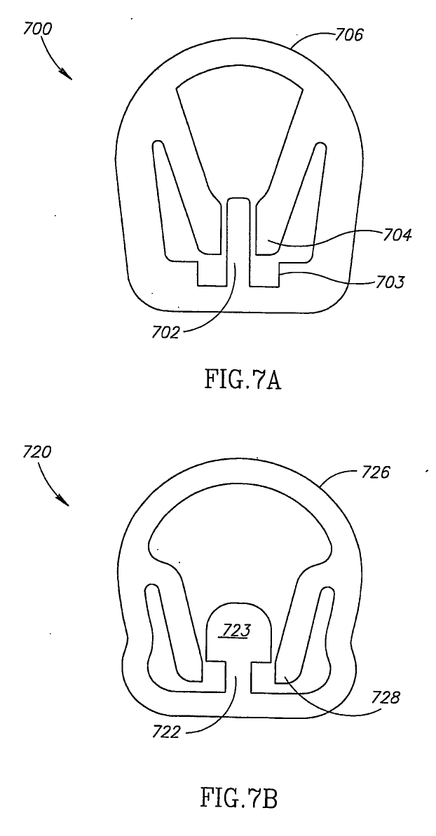

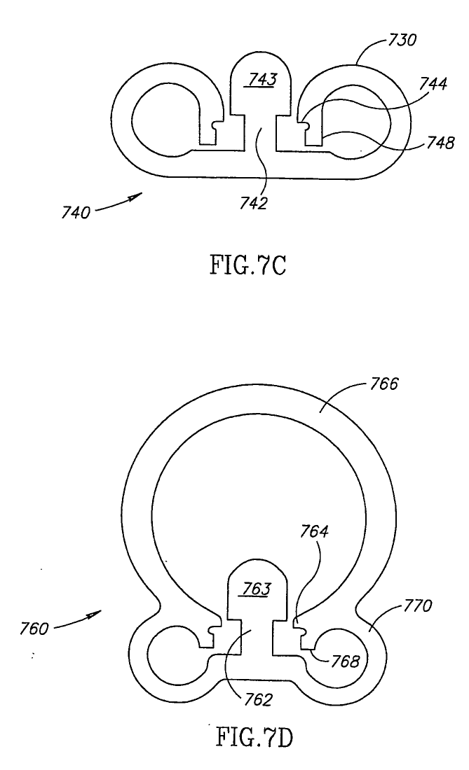

FIGS. 7A-7D shows embodiments of medallion sections that ride on a slot of a hook section, in accordance with exemplary embodiments of the invention;

FIG. 7A shows a medallion 700, in which a tab 702 is adapted to ride inside slot 337, for example. One or more front tabs 704 are optionally provided to urge against the hook section, optionally locking into apertures of the hook section. A ring section 706 optionally also serves as a spring for tabs 704. An alternative locking mechanism is that walls 703 on either side of tab 702 engage a locking area such as area 686.

FIG. 7B shows a medallion 720, in which a tab riding section 722 includes a mushroom extension 723, to prevent it from slipping off slot 637. A pair of side tabs 728 are provided to apply force from the sides of the hook section. Ring 726 optionally provides tabs 728 with some elasticity. Medallion 720 is optionally mounted by distorting extension 723 during mounting.

FIG. 7C shows a medallion 740, which has no ring. Side and/or forward pressure on the hook section are provided by extension sections 744 (forward) and 748 (side), which receive their elasticity from a spring section 750.

FIG. 7D shows a medallion 760 similar to medallion 740 of FIG. 7C, including a ring section 766. In an alternative embodiment, a mushroom section 763 of a riding tab 762 is extended to act as a ring section (e.g., to receive the tip of the hook section and/or to serve as a stop to prevent tissue motion).

Front and/or Back Locking Medallion Variations

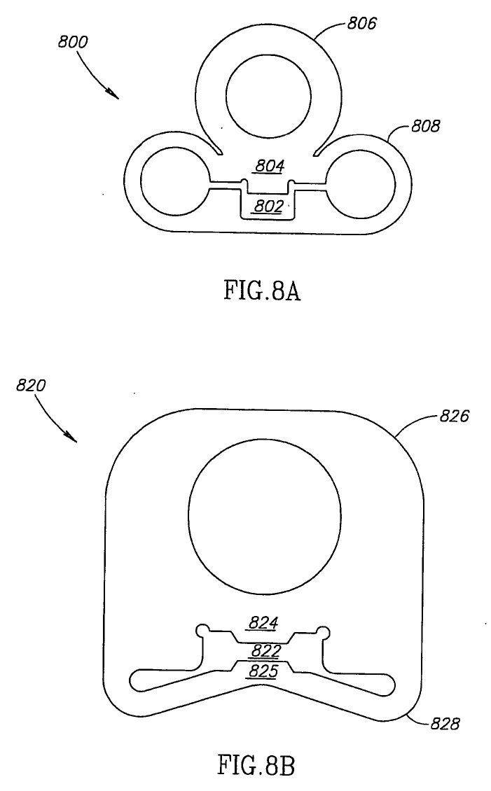

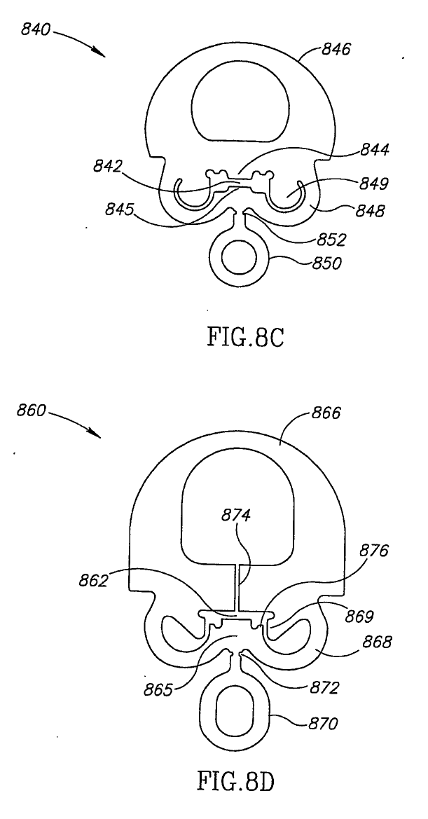

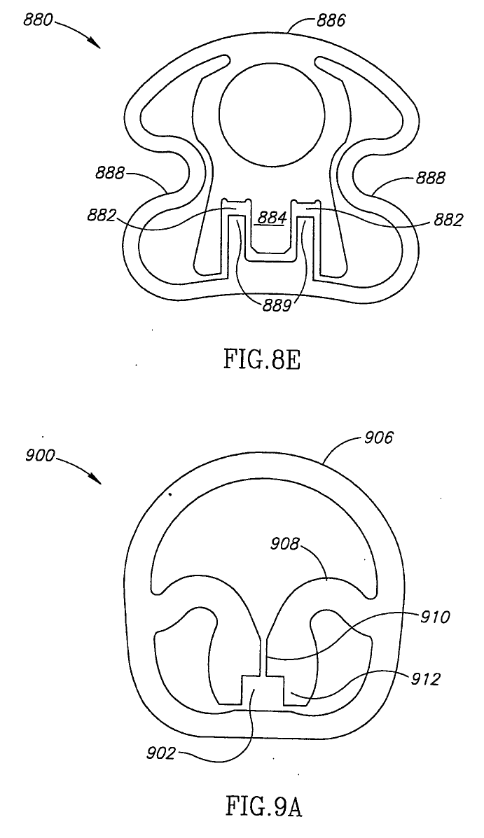

FIGS. 8A-8E show embodiments of medallions with means for locking to a hook section by one or more tabs of the medallion engaging the hook section from its front and/or back side, in accordance with exemplary embodiments of the invention.

FIG. 8A shows a medallion 800, in which a hook section fits in an aperture 802 thereof. When the hook section is to be locked, an aperture of the hook section is moved to be adjacent a forward locking tab 804 of medallion 800 and is urged into the aperture of the hook, by virtue of the original width of aperture 802 being smaller than a thickness of the hook. A spring section 808 optionally provides elasticity for this urging. An optional ring section 806, is shown as well.

FIG. 8B shows an alternative medallion 820, in which both a forward locking tab 824 and a backwards locking tab 825 are provided projecting into an aperture 822 for the hook section. In the embodiment shown, a spring 828 is provided only for backwards locking tab 825. Alternatively or additionally, a spring is provided for tab 824.

FIG. 8C shows an alternative medallion 840, in which a forward locking tab 844 and a backwards locking tab 845 are provided projecting into a hook aperture 842. One or both the tabs may be optional, for example, if the hook section includes a locking tab. An optional side section 849 optionally defines the width of aperture 842. Optionally, section 849 is elastically urged into aperture 842. Optionally, a pull-ring 850 is provided, which can be used to open aperture 842 by pulling on a spring 848 which advances tab 845 into aperture 842. Optionally, pull ring 850 is used during mounting, to widen the aperture. Alternatively or additionally, pull ring 850 is used to suture the medallions together. A weaken section 852 is optionally provided to assist in removing pull ring 852, for example by bending or by tearing.

While a closed ring section 846 is shown, in other embodiments, the ring section is open, for example defining to fingers or arcs away from aperture 842.

FIG. 8D shows an alternative medallion 860, in which only a backwards locking tab 865 is provided. Optionally, however, a slot 874 is provided in a body ring 866, which slot enables ring 866 to provide elasticity for two side tabs 869 that engage the hook section from the sides. Optionally one or more side horns 876 are provided near tab 865, to prevent over advancing of tab 865 into the hook section. Alternatively or additionally, such circular cutouts are provided as part of a cutting design process which takes a minimum cutting radius into account, especially when there is a need for straight lines.

In this variation, a pull-ring section 870 is ellipsoid rather than round.

FIG. 8E shows an alternative medallion 880, in which a forward locking tab 884 is long enough to completely pass through a matching aperture in the hook section. A pair of tabs 889 is optionally provided to urge the hook section onto tab 884. Due to the large distance of motion, in an exemplary embodiment of the invention, a pair of long springs 888 are provided, which attach to the far side of a ring section 886. Optionally, tab 884 extends from ring section 886.

Side Aperture Medallion

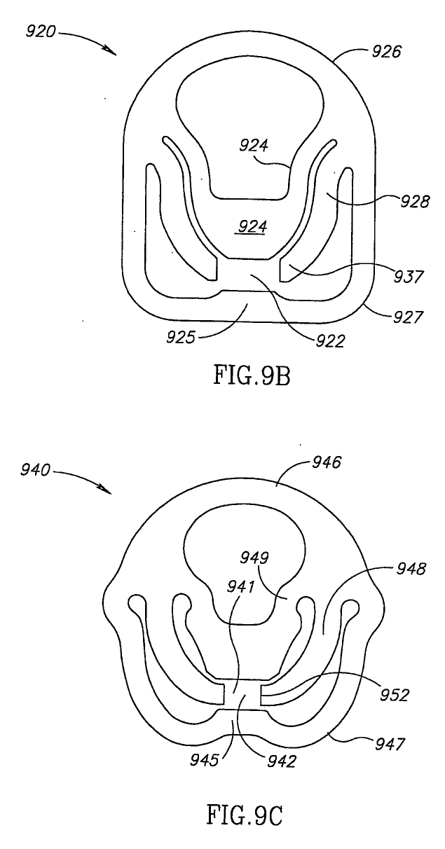

In some embodiments of the invention, locking is provided by a tab of the medallion fitting into a narrowing of the hook section. Connector 400 is an example of this mechanism, in which the “narrowing” is made by providing a widening on either side of the locking area. In alternative embodiments, the width of the hook section is relatively except for a narrowing section. While in connector 400, the medallion section was not required to cooperate, In the embodiments of FIG. 9A-9C, the medallion actively urges tabs into a narrowing (relative or absolute) of the hook section.

FIG. 9A shows a medallion 900 in which a pair of side locking tabs 912 lock into apertures or notches (e.g., a narrowing) defined in a side of a hook section, in accordance with an exemplary embodiment of the invention. A pair of springs 908 are adapted to urge tabs 912 towards an aperture 902 where the hook section rides. Optionally, springs 908 allow tabs 912 to be moved sideways as well as forwards and backwards. This allows the dimensions of aperture 902 to be limited by tabs 912, even before locking. Optionally, a ring section 906 provides some sideways elasticity, which sideways motion is made possible by a slot 910 separating springs 908 from each other.

FIG. 9B shows an alternative medallion 920, in which an aperture 902 for the hook section is defined on either side by a side tab 932, by a front tab 924 and by a back tab 925. Optionally, back tab 925 is elastically urged into aperture 922 by a spring 927. Alternatively or additionally, front tab 924 is elastically urged into aperture 922 by a spring 929. Alternatively or additionally, locking is provided by side tabs 932, which are mounted on springs 928, attached to ring 926.

FIG. 9C shows an alternative medallion 940 similar to that of FIG. 9B, in which a pair of springs 947 that urge a backwards tab 945 into an aperture 942 are more pronounced. Similarly, a pair of springs 949 which urge a front tab 944 into aperture 942 are also more pronounced. In this embodiment, the increased prominence of the springs comprises increased length caused by additional bends, which allow for more elastic motion.

Puller Connector

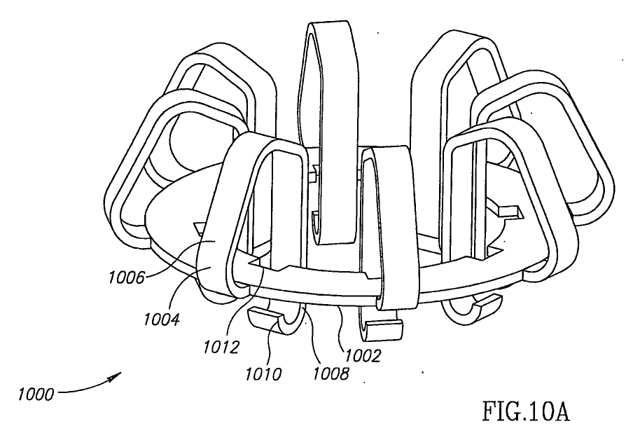

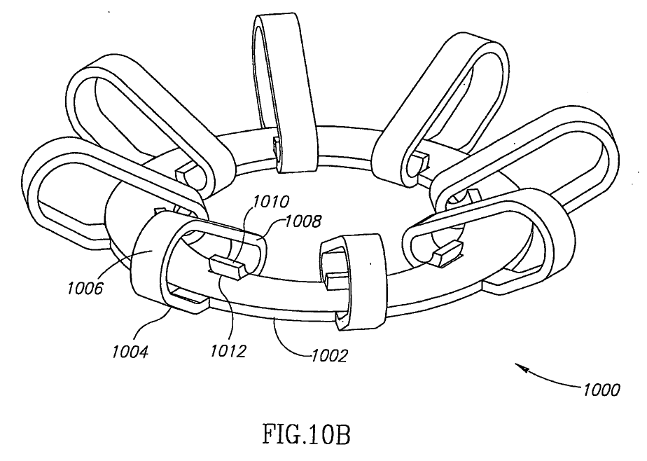

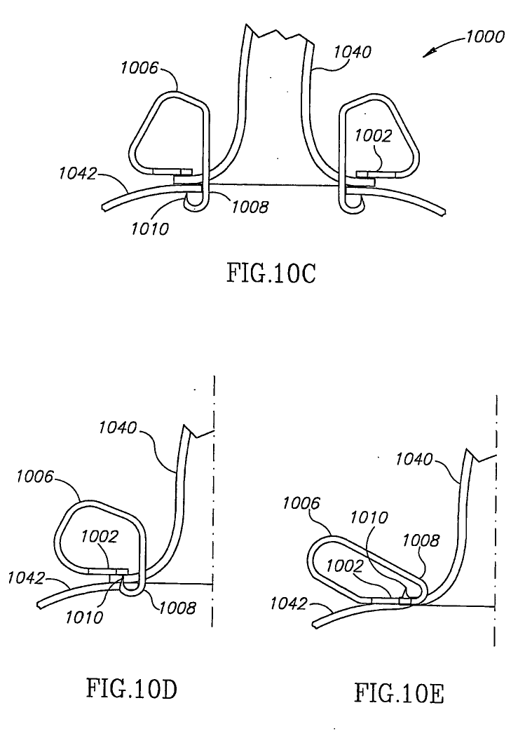

FIGS. 10A-10E illustrate a puller connector 1000 and its use, in accordance with an exemplary embodiment of the invention. FIGS. 10A and 10B show connector 100 in pre-deployed and post-deployed configurations, with no blood vessels attached. In an exemplary embodiment of the invention, connector 1000 is pre-stressed to go from one configuration to the other, for example, being elastic, super elastic or shape memory. Alternatively, it may be plastically deformed to pass between configurations, for example by pulling on looped body sections 1006, described below.

Connector 1000 comprises a ring 1002 to which are attached a plurality of pullers 1004. In an exemplary embodiment of the invention, pullers 1004 have looped bodies 1006 with forward sections 1008 which extend into ring 1002. Forward sections 1008 optionally comprise hooked ends 1010, which may be, for example, adapted to pierce blood vessel tissue with minimum trauma and/or tearing. Alternatively or additionally, at least some of ends 1010 are adapted to not pierce blood vessel tissue, for example being flattened. Alternatively or additionally, at least some of ends 1010 are adapted to pierce tissue only a certain distance, for example by being forked and/or by including tissue stops (not shown) which widen ends 1010.

Optionally a recess 1012 is provided in ring 1002 to receive hook 1010 when puller 1004 is retracted. As shown, the recess is approximately the width of hook 1010. Alternatively, it may be wider, for example to prevent pinching of tissue between hook 1010 and recess 1012.

FIG. 10B shows connector 1000 with pullers 1004 retracted. In an exemplary embodiment of the invention, the length and shape of pullers 1004 allows a relatively long pulling motion to be provided on connector 1000 itself, without requiring a specialized delivery system beyond that required for releasing connector 1000 to pull on pullers 1004. Alternatively or additionally, the shape of pullers 1004 allows a rotational movement to be carried out by hooks 1010. Optionally, a ring 1002 is not provided and is either dispensed with or provided as part of the delivery system. In this case, the base sections of pullers 1004 are optionally made wide, so that they apply force to a relatively large section of the blood vessel with which it is in contact. Alternatively, ring 1002 is made radially expandable, for example, using expandable cell sections between adjacent pullers.

It should be noted that FIG. 10B shows hooks 1010 lying inside of recesses 1012. However, if recesses 1012 are not wider than shown, the existence of vascular tissue between hook 1010 and recesses 1012 may prevent such a configuration. Alternatively, if hooks 1010 pierce the vascular tissue, such configuration, as shown, is possible, with all of the vascular tissue lying within ring 1002.

FIGS. 10C-10E show the deployment of connector 1000, in one design variation thereof, where the pullers are not substantially rotated by the pulling. In some cases, the hooks rotate, for example, 10 degrees, 30 degrees, 50 degrees or any intermediate or large amount of rotation.

In FIG. 10C, a graft vessel 1040 is everted and mounted on hooks 1010 and hooks 1010 are inserted into a target vessel 1042. The delivery system is optionally retracted to engage target vessel 1042 by hooks 1010.

In FIG. 10D, hooks 1010 have retracted sufficiently to pierce target vessel 1042 and to push vessel 1040 hard enough against ring 1002 so that graft vessel 1040 is also pierced by hooks 1010 (a second time). if the everted section of graft vessel 1040 is short enough, no second piercing will occur.

FIG. 10E shows an optional further retraction configuration, in which hooks 1010 pass through recesses 1012 (not shown) in ring 1002.

The following documents, the disclosures of which are incorporated herein by reference describe connectors, delivery systems and/or other tools and methods which are useful in conjunction with embodiments of the prevent invention:

PCT/IL02/00790, filed on Sep. 25, 2002, now published as WO 03/026475;

U.S. Ser. No. 60/492,998 filed on Aug. 7, 2003.

PCT/IL02/00215, filed on Mar. 18, 2002, now published as WO 02/074188;

PCT/IL01/01019, filed on Nov. 4, 2001, now published as WO 02/47532;

PCT/IL01/00903, filed on Sep. 25, 2001 now published as WO 02/30172;

PCT/IL01/00600, filed on Jun. 28, 2001, now published as WO 02/47561;

PCT/IL01/00267, filed on Mar. 20, 2001, now published as WO 01/70091;

PCT/IL01/00266, filed on Mar. 20, 2001, now published as WO 01/70090;

PCT/IL01/00074, filed on Jan. 25, 2001, now published as WO 01/0119;

PCT/IL01/00069, filed on Jan. 24, 2001, now published as WO 01/70118;

PCT/IL00/00611, filed on Sep. 28, 2000, now published as WO 01/41624;

PCT/IL00/00609, filed on Sep. 28, 2000, now published as WO 01/41623,

PCT/IB00/00310, filed on Mar. 20, 2000, now published as WO 00/56228;

PCT/IB00/00302, filed on Mar. 20, 2000, now published as WO 00/56227;

PCT/IL99/00674, filed on Dec. 9, 1999, now published as WO 00/56223;

PCT/IL99/00670, filed on Dec. 8, 1999, now published as WO 00/56226;

PCT/IL99/00285, filed on May 30, 1999, now published as WO 99/62408; and

PCT/IL99/00284, filed on May 30, 1999, now published as WO 99/62415. The disclosure of all of these applications, which designate the US and were filed in English, are incorporated herein by reference.

In addition, a PCT application filed on same date with the present application, by applicant “By-Pass Inc.”, and describing anastomotic connectors is “Sliding Surgical Clip”, attorney docket number 088/03506, the disclosure of which is incorporated herein by reference. A PCT application filed on same date with the present application, by applicant “By-Pass Inc.”, and describing leg arranging systems is “Anastomotic Leg Arrangement”, attorney docket number 088/03504, the disclosure of which is incorporated herein by reference.

A provisional application filed on even date with the instant application, by applicants Loshakove, et. al and having attorney docket number 088/03695 and title “Bypass Punch Anastomosis Delivery System” is also incorporated herein by reference and describes an exemplary delivery system.

A clip or a connector may be manufactured of various materials, including for example, metals (e.g., stainless steel, NiTi alloys and titanium), plastics and bio-absorbable materials. Optionally, the clip is formed of a material that exhibits elastic, super elastic and/or shape memory properties.

Some of these applications describe anastomosis delivery systems and hole making apparatus and/or other device useful in cooperation with the present invention. Some of these applications describe delivery systems in which separate steps are provided for retracting and tearing, and even, in some embodiments, for advancement of medallion sections.

The above described clips and connectors and their use may be varied in many ways. For example, the hook sections and/or the medallions may be interconnected before or after the anastomosis, for example, using a flexible element, such as a suture, or a rigid element, such as a metal bar.

In an exemplary embodiment of the invention, the tips that are designed to penetrate blood vessel tissue are sharpened to minimize trauma to the blood vessels, during attaching, and especially to reduce tearing and/or dissection. For example, the tips may be formed to be needle like, so that they have no edges that can tear nearby tissue, except when inserted, tip first. Such forming may be, for example, by electro-polishing.

While the above clips have been described in general for any type of blood vessel, it should be appreciated that particular modifications may be desired for certain vessel types. For example, the aorta is thicker, while a coronary vessel is thinner, thus suggesting different amounts of space in the clip. For example, an aorta may be 3 mm thick, while a coronary vessel may be less than 1 mm thick.

In an exemplary embodiment of the invention, clip 100 is between 0.17 mm and 0.2 mm thick, is 0.4 mm wide on its body and 0.8 mm wide at its base and curves in a radius of 0.8 mm. With reference to FIG. 4, a space between the hook section and the medallion after locking can be, for example, 0.25 mm. The hook can be, for example, between 0.15 and 0.17 mm thick and between 0.29 and 0.35 mm wide. The medallion can be, for example, 0.4 mm thick with an area about 0.8 mm by 0.6 mm. These sizes are only exemplary and are intended to give a measure of the size of the elements involved. Each such dimension can be, for example, twice as large or half the size, depending on the particular application

It should be noted that the term “connector” should be construed broadly to include various types of connectors, including one part, two part and multiple part connectors, some of which when deployed, result in a plurality of individual clip-like sections.

The term “eversion”, where used means not only complete eversion of 180 degrees, but also partial eversion or flaring, for example of 90 degrees. Also, in some embodiments, mounting without eversion is provided.

Measurements are provided to serve only as exemplary measurements for particular cases. The exact measurements stated in the text may vary depending on the application, the type of vessel (e.g., artery, vein, xenograft, synthetic graft), size of connector, shape of hole (e.g., incision, round) and/or sizes of vessels involved (e.g., 1 mm, 2 mm, 3 mm, 5 mm, aorta sized).

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A self-locking clip adapted for vascular tissue connection, comprising:
a body;
a sharp extension on one side of the body adapted to pierce blood vessel tissue; and
a base on another side of said body, said base adapted to interlock with said extension, at least to prevent retraction of said extension from said base after inserted,
wherein said clip is pre-disposed to assume a closed configuration where said base locks to said extension.

2. A clip according to claim 1, wherein said extension defines a plurality of locking positions.

3. A clip according to claim 1, wherein said base defines an aperture adapted to receive said extension.

4. A clip according to claim 3, wherein said aperture is adapted to guide said extension to be locked.

5. A clip according to claim 1, wherein said clip is adapted to be used as part of a set of a plurality of clips to complete single anastomosis connection.

6. A clip delivery system, comprising:
a plurality of clips adapted for vascular tissue connection, each clip comprising:
a body;
a sharp extension on either end of said body,
wherein said clip is pre-disposed to form a “C” shape;
an inner tube defining a plurality of slots; and
an outer tube axially movable with respect to said inner tube,
wherein said inner and outer tube define a receptacle for said clip, said clip being released when said outer tube is retracted relative to said inner tube and wherein said inner tube defines a slot adjacent said receptacle, said slot adapted to receive a bent-back section of a backwards pointing one of said extensions.

7. A delivery system according to claim 6, wherein said body defines an aperture and wherein said inner tube defines a matching protrusion to said aperture and wherein said body curves when released, such that said aperture is released from said protrusion.

8. A connector clip adapted for vascular tissue connection, comprising:
a hooked element comprising:
an elongate body;
a designated tear location structurally defined at a location along said body;
a hooked tip adapted to pierce a blood vessel; and
a base element adapted to lock to said hooked element,
wherein said hooked element is adapted to not tear vascular tissue.

9. A clip according to claim 8, wherein said hooked tip is adapted to not cut vascular tissue.

10. A clip according to claim 8, wherein said hooked tip has the shape of a needle.

11. A clip according to claim 8, wherein said hooked tip is manufactured by cutting and smoothing a planar material.

12. A connector clip adapted for vascular tissue connection, comprising:
a hooked element comprising:
an elongate body;
a designated tear location structurally defined at a location along said body;
a designated locking location structurally defined at a location along said body;
a hooked tip adapted to pierce a blood vessel; and
a base element adapted to lock to said hooked element at said locking location,
wherein said designated tearing location is configured to enhance a locking at said locking location.

13. A clip according to claim 12, wherein said tearing location is configured so that tearing causes the bending of at least one part of said hooked element to narrow a passage of a portion of said base element within said hooked element.

14. A clip according to claim 12, wherein said tearing location is configured so that tearing causes the bending of at least one part of said hooked element to widen a portion of said hooked element which travels within said base element.

15. A clip according to claim 12, wherein said tip is adapted to pierce vascular tissue without causing tearing.

16. A connector clip adapted for vascular tissue connection, comprising:
a hooked element comprising:
an elongate body having a slot defined therein;
a designated tear location structurally defined at a location along said body;
a designated locking location structurally defined at a location along said body;
a hooked tip adapted to pierce a blood vessel; and
a base element adapted to lock to said hooked element at said locking location and including a section that fits in said slot,
wherein said designated locking location is located in said slot.

17. A clip according to claim 16, wherein said hooked element comprises a second designated locking location on an outside of said hooked element.

18. A clip according to claim 16, wherein said tip is adapted to pierce vascular tissue without causing tearing.

19. A clip according to claim 16, wherein said base element is planar.

20. A connector clip adapted for vascular tissue connection, comprising:
a hooked element comprising:
an elongate body;
a designated tear location structurally defined at a location along said body;
a designated locking location structurally defined at a location along said body;
a hooked tip adapted to pierce a blood vessel; and
a base element adapted to lock to said hooked element at said locking location,
wherein said designated locking location is defined by at least one active portion on said hooked element which engages a portion of said base section.

21. A connector according to claim 20, wherein said base element includes no portions that move relative to a center of gravity of said base element during a locking activity.

22. A connector according to claim 20, wherein said base element is planar.

23. A clip according to claim 20, wherein said tip is adapted to pierce vascular tissue without causing tearing.

24. A connector clip adapted for vascular tissue connection, comprising:
a hooked element comprising:
an elongate body;
a designated tear location structurally defined at a location along said body;
a designated locking location structurally defined at a location along said body;
a hooked tip adapted to pierce a blood vessel; and
a base element defining a second locking location adapted to lock to said hooked element, wherein both said base element and hooked element each include at least one portion that moves during locking.

25. A connector clip adapted for vascular tissue connection, comprising:
a hooked element comprising:
an elongate body having an axis;
a designated locking location structurally defined at a location along said body;
a hooked tip adapted to pierce a blood vessel; and
a base element having an aperture adapted to ride on said body, said base element adapted to lock to said hooked element at said designated locking location,
wherein said locking location comprises at least one widening on said hooked element perpendicular to said axis and at least one aperture formed in said body adjacent said widening.

26. A connector clip adapted for vascular tissue connection, comprising:
a hooked element comprising:
an elongate body having an axis;
a hooked tip adapted to pierce a blood vessel; and
a base element having an aperture adapted to ride on said body and wherein at least one section of said base element contacting said aperture is adapted to be elastically moved to widen said aperture and comprising:
at least one holder adapted to widen said aperture when pulled against a resistance of said hooked element in said aperture.

27. A connector according to claim 26, wherein said holder comprises an apertured holder.

28. A connector according to claim 27, wherein said aperture contains a thread.

29. A connector according to claim 26, wherein said holder is adapted to be torn off said base element.

30. A method of mounting a base element of an anastomotic clip on a hooked element of an anastomotic clip, comprising:
placing said hooked element in an aperture of said base element; and
pulling on a holder section of said base element to widen said aperture.

31. A method of demounting a base element of an anastomotic clip on a hooked element of an anastomotic clip, comprising:
pulling on a holder section of said base element to widen an aperture of said base element on which said hooked element is mounted; and
removing said base element.

32. A connector clip set adapted for performing a vascular anastomotic connection, comprising:
a plurality of connector clips, each comprising:
a hooked element comprising:
an elongate body having an axis, a first end and a second end;
a pulling point adapted to have a pulling force applied to at said first end
a hooked tip adapted to pierce a blood vessel at said second end
a resting point for a base element between said ends; and
a base element adapted to ride on said body and stop at said resting point,
wherein a distance between said resting point and said pulling point is different for different ones of said clips.

33. A connector according to claim 32, wherein said resting point is adapted to withstand a force of at least 1 Kg applied from said pulling point.

34. A pulling connector adapted for vascular tissue connection, comprising:
a ring; and
a plurality of hooked elements, extending from the ring, having an elongate body curved into said ring,
wherein said elongate elements are pre-disposed to retract such that they pull vascular tissue towards said ring to complete an anastomotic connection.

35. A connector according to claim 34, wherein said hooked elements rotate when released.

36. A connector according to claim 34, wherein said curves flatten when released.

37. A connector according to claim 34, wherein said hooked elements are adapted to pierce blood vessel tissue without tearing.

38. A connector according to claim 34, wherein said ring defines a plurality of recesses for said hooked elements.

39. A connector according to claim 34, wherein said ring is substantially rigid.

40. A method of deploying a tearing vascular anastomotic connector having multiple tearing points, comprising:
tearing a first leg to complete a first part of an anastomosis; and
tearing a second leg after said first tearing to complete a second part of said anastomosis.

41. A method according to claim 40, comprising applying a continuous force to said connector during said first and second tearing and in between.
